(19) United States
(12) Patent Application Publication
(10) Pub. No.: US 2022/0344026 A1
(43) Pub. Date: Oct. 27, 2022

(54) INTERACTION BETWEEN A MASK AND A PATIENT DURING THERAPY

(71) Applicant: KONINKLIJKE PHILIPS N.V., EINDHOVEN (NL)

(72) Inventors: Cornelis Petrus Hendriks, Eindhoven (NL); Joyce van Zanten, Eindhoven (NL); Pedro Miguel Ferreira dos Santos da Fonseca, Eindhoven (NL); Joachim Johannes Kahlert, Aachen (NL); Olaf van der Sluis, Eindhoven (NL)

(21) Appl. No.: 17/725,918

(22) Filed: Apr. 21, 2022

Related U.S. Application Data
(60) Provisional application No. 63/180,181, filed on Apr. 27, 2021.

(51) Int. Cl. G16H 20/30; G16H 40/67

(52) U.S. Cl. CPC G16H 20/30; G16H 40/67

(57) ABSTRACT

A system for monitoring the interaction of a mask with a patient's face in real-time during a pressure therapy treatment session and providing recommendations for improving such interaction. A sensing arrangement obtains real-time relative position data regarding the relative position of the mask relative to face of the patient and a computing arrangement in communication with the sensing arrangement for receiving the real-time relative position data from the sensing arrangement. The computing arrangement includes a computational model that uses the real-time relative position data to determine information regarding the interaction between the mask and the face of the patient, and an interpretation and analysis unit that: relates the information regarding the interaction between the mask and the face of the patient to functional parameters, determines actionable information and/or insights to improve the interaction, and provides as output the actionable information and/or insights.

### CROSS-REFERENCE TO RELATED APPLICATIONS

This patent application claims the priority benefit under 35 U.S.C. § 119(e) of U.S. Provisional Application No. 63/180,181, filed on Apr. 27, 2021, the contents of which are herein incorporated by reference.

### BACKGROUND OF THE INVENTION

### 1. Field of the Invention

The present invention pertains to systems and methods for improving the interaction between a mask and a patient when the patient is receiving a positive airway pressure (PAP) treatment via the mask.

### 2. Description of the Related Art

Many individuals suffer from disordered breathing during sleep. Sleep apnea is a common example of such sleep disordered breathing suffered by millions of people throughout the world. One type of sleep apnea is obstructive sleep apnea (OSA), which is a condition in which sleep is repeatedly interrupted by an inability to breathe due to an obstruction of the airway; typically the upper airway or pharyngeal area. Such obstruction of the airway is generally believed to be due, at least in part, to a general relaxation of the muscles which stabilize the upper airway segment, thereby allowing the tissues to collapse the airway. Another type of sleep apnea syndrome is a central apnea, which is a cessation of respiration due to the absence of respiratory signals from the respiratory center of the brain. An apnea condition, whether obstructive, central, or mixed (i.e., a combination of obstructive and central), is defined as the complete or near cessation of breathing, for example a 90% or greater reduction in peak respiratory air-flow.

Those afflicted with sleep apnea experience sleep fragmentation and complete or nearly complete cessation of ventilation intermittently during sleep with potentially severe degrees of oxyhemoglobin desaturation. These symptoms may be translated clinically into extreme daytime sleepiness, cardiac arrhythmias, pulmonary-artery hypertension, congestive heart failure and/or cognitive dysfunction. Other consequences of sleep apnea include right ventricular dysfunction, carbon dioxide retention during wakefulness, as well as during sleep, and continuous reduced arterial oxygen tension. Sleep apnea sufferers may be at risk for excessive mortality from these factors as well as by an elevated risk for accidents while driving and/or operating potentially dangerous equipment.

Even if a patient does not suffer from a complete or nearly complete obstruction of the airway, it is also known that adverse effects, such as arousals from sleep, can occur where there is only a partial obstruction of the airway. Partial obstruction of the airway typically results in shallow breathing referred to as a hypopnea. A hypopnea is typically defined as a 50% or greater reduction in the peak respiratory air-flow. Other types of sleep disordered breathing include, without limitation, upper airway resistance syndrome (UARS) and vibration of the airway, such as vibration of the pharyngeal wall, commonly referred to as snoring.

One of the therapy options for patients suffering from obstructive sleep apnea (OSA) is positive airway pressure (e.g., CPAP, BiPAP, Auto-PAP). In such therapy a device provides a pressurized air flow to the airways via a face or nasal mask. Maintaining therapy compliance is challenging and crucial to achieve good therapy outcomes. A key factor is to maintain a good mask comfort, seal and stability such that the patient is not disturbed in his or her sleep and the device functions properly. However, this is not trivial. Despite a good initial mask fit by a health care provider, things might go wrong at home such as improper strap positioning and tightening, external forces on the mask while tossing and turning, mask wear out, etc. This often causes problems such as high skin pressures, red marks, skin breakdown, leakage, slipping etc., which might directly or indirectly impair the therapy compliance. Hints for what is causing problems can be obtained from distant observations such as leakage detection with sensors in the CPAP machine, or visible facial red marks. However, contextual/situational and local information is missing and hence, there is insufficient insight in the root cause(s) of this problem (when, how, what, where). Knowing root cause(s) is valuable in knowing why the therapy works or does not work, what disturbs the patient or why he or she is not compliant. Hence, there is a need to know when and how something goes wrong in the skin-mask interface, such that an early warning can be provided, and the physician can properly intervene.

Integration of a sensor in the seal or cushion to directly measure the interaction (e.g., pressure, friction, gap) between the mask and the face seems an obvious solution, but this will cause a physical contradiction because the sensor will impair the mask seal or cushion function, even with a thin and flexible piezoelectric film. Also, the integration of a sensor in a mask seal or cushion is difficult, and the mask would become technically very complex and costly. Ecosystem sensors like sleep tracking cameras, mobile devices or wearables offer low cost, continuous and ubiquitous sensing, but such sensors do not have a direct access to the interface between the mask and the face.

Therefore, monitoring mask comfort, seal, and stability in an inexpensive and accurate manner has not yet been solved.

### SUMMARY OF THE INVENTION

Embodiments of the present invention overcome shortcomings of the prior art by providing arrangements that monitor the interaction of a mask with the face of a patient in real-time during a PAP treatment session and provide feedback for improving the interaction between the mask and the patient when the patient is receiving further PAP treatment via the mask.

As one aspect of the invention, a system for monitoring the interaction of a mask with the face of a patient in real-time during a pressure therapy treatment session and providing recommendations for improving such interaction is provided. The system comprises: a sensing arrangement structured to obtain real-time relative position data regarding the relative position of the mask relative to face of the patient; and a computing arrangement in communication with the sensing arrangement for receiving the real-time relative position data from the sensing arrangement. The computing arrangement comprises: a computational model structured to use the real-time relative position data to determine information regarding the interaction between the mask and the face of the patient, and an interpretation and analysis unit structured to: relate the information regarding the interaction between the mask and the face of the patient to functional parameters, to determine actionable information and/or insights to improve the interaction, and to provide as output the actionable information and/or insights.

The system may further comprise a user device executing an application for receiving the output from the interpretation and analysis unit. The user device may comprise a smartphone.

The information regarding the interaction between the mask and the face of the patient may comprise one or more of: a skin pressure distribution resulting from the mask being pressed against the face of the patient, a gap distribution of a number of gaps between the mask and the face of the patient, and/or a slip distribution of the mask relative to the face of the patient.

The functional parameters may comprise one or more of: mask comfort, mask seal, and/or mask stability.

The sensing arrangement may comprise a camera system. The sensing arrangement may comprise a number of physical sensors. The sensing arrangement may comprise a number of thermal sensors. The sensing arrangement comprises a number of microphones.

The computational model may utilize a virtual model of the mask that is positioned, based on the real-time relative positon data, with respect to a virtual head and/or face model that is specific to the patient.

The interpretation and analysis unit may comprise a neural network.

The computing arrangement may be a cloud-based computing arrangement.

As another aspect of the invention, a method for improving the interaction between of a mask used in a pressure therapy treatment session and a face of a patient receiving the pressure therapy is provided. The method comprises: obtaining real-time relative position data of the mask relative to the face of the patient while the patient is receiving the pressure therapy via the mask; determining information regarding the interaction between the mask and the face of the patient; determining one or more of: comfort, seal, and/or stability of the mask that can be improved is determined from the information regarding the interaction; and determining actionable information to improve the one or more of comfort, seal, and or stability of the mask on the face of the patient.

The method may further comprise one or both of: providing the actionable information to the patient or a caregiver of the patient; and/or carrying out the actionable information.

Determining information regarding the interaction between the mask and the face of the patient may comprise determining one or more of: a skin pressure distribution, a gap distribution, and/or a slip distribution.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic representation of a system in accordance with one example embodiment of the present invention shown monitoring an example PAP treatment session being provided via a mask to the airway of a patient; and

FIG. 2 is a flow chart showing general steps of a method in accordance with one example embodiment of the present invention.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

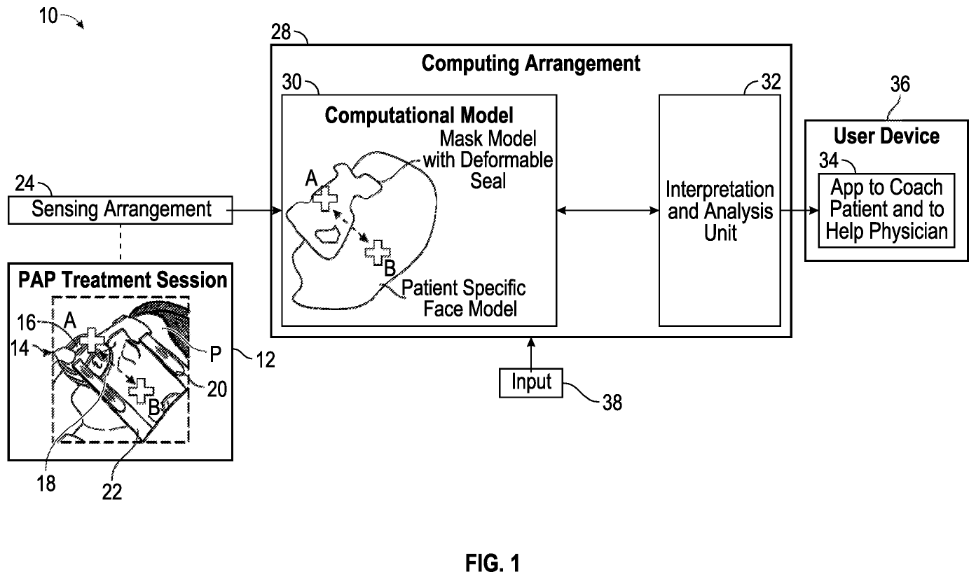

As discussed above, embodiments of the present invention provide arrangements that monitor the interaction of a mask/patient interface with the face of a patient in real-time during a PAP treatment session and provide feedback from such monitoring for improving the interaction between the mask/patient interface and the face of the patient when the patient is receiving further PAP treatment via the mask/patient interface. Such improvements may include, for example, without limitation, improved comfort, seal, and/or stability between the mask and the face of the patient, any/all of which typically lead to improved compliance with the PAP therapy. A schematic representation of a system 10 in accordance with one example embodiment of the present invention for carrying out such task is shown in FIG. 1 monitoring an example PAP treatment session 12 (e.g., CPAP, BiPAP, Auto-PAP) being provided via a mask 14 to a patient P (it is noted that an associated pressure generating device providing the pressurized treatment gas is not shown in FIG. 1).

In the example shown in FIG. 1, mask 14 is shown as a oronasal mask (i.e., covers the nose and the mouth of patient P) having a generally rigid shell 16 bounded by a soft, conformable cushion 18 that is compressed against the face of patient P via upper and lower headgear straps 20 and 22. It is to be appreciated, however, that mask 14 is provided for exemplary purposes only and that system 10 may be utilized in monitoring PAP treatment sessions provided via other types of masks/patient interfaces (e.g., without limitation, nasal masks, full face masks, nasal pillows, etc.) secured via any suitable strapping arrangements or headgear without varying from the scope of the present invention.

Continuing to refer to FIG. 1, system 10 includes a sensing arrangement 24 positioned in proximity to PAP treatment session 12 for carrying out the aforementioned monitoring of the interaction of mask 14 with the face of patient P during PAP treatment session 12. More particularly, sensing arrangement 24 continuously monitors the positioning of mask 14 and the head/face of patient P. As discussed below in conjunction with several example embodiments, such monitoring by sensing arrangement 24 may be carried out using various techniques without varying from the scope of the present invention. During such monitoring, real-time relative position data regarding the positioning of mask 14 relative to the head of patient P is obtained by sensing arrangement 24 and communicated via any suitable wired or wireless arrangement to a computing arrangement 28 that is also included as a component of system 10. From such relative position data particular details (discussed in detail below) of the interface between mask 14 and the face of patient P can be determined.

Computing arrangement 28 includes a number of suitable processing devices (not numbered) and associated memory storage arrangements (not numbered) as needed for the particular application. Additionally, computing arrangement 28 may include a predictive AI system, such as a trained neural network or other supervised learning systems that is/are structured to receive the relative position data as well as various other data related to the patient, the PAP treatments provided thereto, as well as any other suitable data, and to provide suggestions based on such data for improving PAP treatment provided to the patient. Computing arrangement 28 may be local to sensing arrangement 24 or alternatively may be a cloud-based computing arrangement remote from sensing arrangement 24 and accessed via a suitable wired or wireless internet connection, or other suitable arrangement.

Computing arrangement 28 includes a computational model 30 for receiving and utilizing the real-time relative position data from sensing arrangement 24 and determining therefrom time-based information regarding the interaction between mask 14 and the face of patient P. In the example shown ion FIG. 1, computational model 30 is structured/programmed to use the real-time relative position data obtained by, and communicated from sensing arrangement 24 to evaluate one or more of: skin pressure distribution resulting from mask 14 being pressed against the face of patient P, gap distribution (i.e., space(s) between mask 14 and the face of patient P), and/or slip/shear distribution (i.e., misalignment(s)) of mask 14 relative to the face of patient P over time. In one example embodiment, such evaluation by computational model 30 is accomplished by comparing a virtual model of the particular mask being employed as mask 14 that is overlaid/positioned, based on the real-time relative positon data received from sensing arrangement 24, on a virtual head/face model specific to patient P. A benefit of computational model 30 is that it translates the real-time relative position data into the aforementioned local skin pressure, gap, and slip data that cannot be acquired in another way without impairing the mask function. Hence, the synergy between the real-time relative position data and computational model 30 enables monitoring of mask comfort, seal and stability in a generally inexpensive, continuous and unobtrusive way.

Computing arrangement 28 further includes an interpretation and analysis unit 32 (e.g., a predictive artificial intelligence (AI) system, such as the trained neural network or other supervised learning systems previously discussed) that is structured/programmed to relate the computational results determined in, and provided by, computational model 30 to functional parameters (e.g., mask comfort, mask seal, mask stability) and to provide as an output actionable information and/or insights in the form of warnings and/or recommendations to the patient, physician, or other appropriate person(s) regarding the monitored PAP treatment session so as to improve the interaction between mask 14 and patient P, and thus improve the PAP therapy and compliance thereto. Alternatively, the actionable information provided by analysis unit 32 may improve the PAP therapy by providing a suggestion to replace mask 14 with another mask like mask 14 (e.g., due to the original mask 14 being past a useful life) or with a different mask likely better suited for patient P (e.g., different size, style, arrangement for securing, etc.). Such output may be provided via a software application 34 executed on a user device 36 (e.g., a smartphone, tablet computer, laptop computer, or any other suitable device) included and/or associated with system 10 via any suitable wired or wireless connection. As will be appreciated from the various example embodiments discussed below, computing arrangement 28 may also consider/utilize various other input(s) 38 provided thereto in formulating the actionable information and/or insights provided the patient , physician, etc.

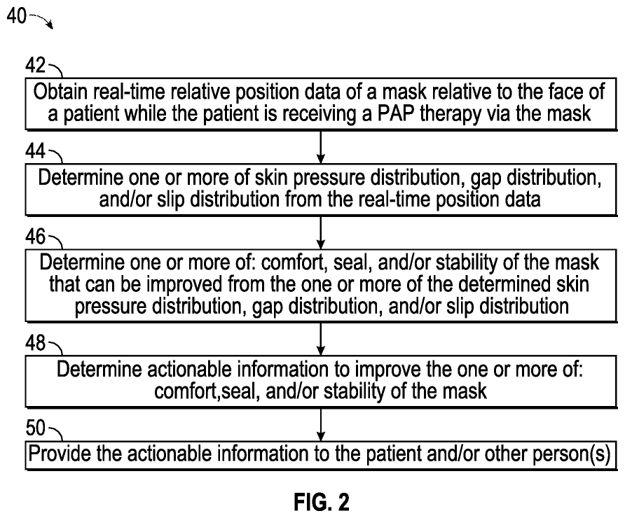

Having thus described a system 10 in accordance with one example embodiment of the present invention, a method 40 for improving the interaction between a mask and a patient when the patient is receiving a positive airway pressure (PAP) treatment via the mask which may be carried out using system 10 will now be described in reference to FIG. 2 as well as FIG. 1. Method 40 begins at 42 where real-time relative position data of mask 14 relative to the face of patient P is obtained (e.g., via sensing arrangement 24) while patient P is receiving a PAP therapy via mask 14. From the real-time position data, information regarding the interaction between mask 14 and the face of patient P is determined. For example, as shown at 44, one or more of: a skin pressure distribution, gap distribution, and/or slip distribution may be determined from the real-time position data obtained in 42.

Next, as shown at 46, one or more of: comfort, seal, and/or stability of the mask that can be improved is determined from the one or more of the skin pressure distribution, gap distribution, and/or slip distribution determined in 44. Next, actionable information to improve the one or more of comfort, seal, and or stability of the mask is determined at 48 and subsequently provided to the patient and/or other person(s) (e.g., caregiver, doctor, and/or any other appropriate individual or individuals), such as shown at 50. After receiving the actionable information, it is up to the patient/caregiver, etc. to carry out the suggested course(s) of action to improve the interaction of mask 14 with the face of patient P and thus improve the PAP treatment.

Having thus described both a system 10 and method 40 in accordance with general example embodiments of the present invention, some further detailed examples of more particular embodiments will now be discussed with continued reference to FIG. 1.

In one example embodiment in accordance with the present invention, the required model input to computational model 30 is the location and the orientation of mask 14 relative to the head of patient P in 6 degrees of freedom (DOF), e.g., position and angular position relative to each of the x, y and z axis of mask 14 in regard to the face of patient P. This information can be obtained in different ways using known sensor technologies. As a reference, the best (i.e., optimum) position of mask 14 relative to the face/head of patient P may, for instance, be established during a visit to a sleep lab, DME or hospital. The actual position of mask 14 may be determined using a camera system as sensing arrangement 24 in combination with image processing techniques, such as known imaging systems presently used in robotics and virtual or augmented reality which can determine the location and orientation of objects in a 3D space. In such example embodiment, the location and orientation of both the head of patient P and mask 14 is determined, for example, with a combination of anatomical and artificial markers (nose, ears, eyes, mask shell 16, tube connection, forehead pad etc.) and/or physical markers (e.g. stickers on mask shell 16). This can provide the required model input.

It is anticipated that 6 DOF cameras and image processing techniques will become better and affordable in the next decade. Currently however, 6 DOF pose estimation is still challenging. Accordingly, in another example embodiment in accordance with the present invention a combination of a 2D camera system and a physical sensor separate from the camera system is utilized as sensing arrangement 24. In such example, the 3 DOF head and mask orientation is measured with a 2D camera and predetermined 3D head and mask models using a method such as described in “Single image-based head pose estimation with spherical parametrization and 3D morphing”, Yuan H. et al., Pattern Recognition 103 (2020) 107316 9 (the contents of which are incorporated by reference herein) and the relative mask position is measured with a contactless physical sensor, e.g., of the type capacitive, photoelectric, ultrasonic, etc., or with a stretch or force sensor in the head gear. 3D geometrical head models of patients may be obtained with the Philips mask fit solution or any other suitable arrangement and 3D mask models from CAD files or any other suitable sources.

As an alternative, if a camera system such as previously described is not available, the location and position of mask 14 relative to the head of patient P can be determined using a combination of physical sensors that can measure distance and orientation without contact, such as sensor types commonly used in mobile phones (e.g., proximity, accelerometer, gyroscope, optical). These sensors can be integrated in mask 14, albeit generally away from the portion(s) thereof interacting with the face of patient P so as to avoid interfering with such interaction as previously discussed. Such arrangements utilizing multiple sensors integrated with mask 14 are presently less preferred due to the added complexity in the mask design as well as additional calibration procedures typically needed.

In another example embodiment, a single proximity sensor may be integrated in mask 14 to measure the distance between mask 14 and the nose, forehead, or other suitable landmark of patient P for example in 1 DOF (and thus provide a distance between known reference points on each of mask 14 and patient P, such as points A and B in FIG. 1). While such sensor does not measure the orientation of mask 14, if it is assumed that mask 14 is generally properly aligned (i.e., no slip) with a symmetrical mask fit, such arrangement can provide a first order estimation of the relative positioning of mask 14 with respect to the face of patient P as well as trend information related thereto. Sensor technologies for quantitatively sensing the distance between a surface and a human object that may be employed in such embodiment are known and are being developed for human-robot collaborations, see e.g., Tsuji, S. and Kohama, T., 2020, “Proximity and Contact Sensor for Human Cooperative Robot by Combining Time-of-Flight and Self-Capacitance Sensors”, IEEE SENSORS JOURNAL, VOL. 20, NO. 10, MAY 15, 2020—the contents of which are incorporated by reference herein. Two or more proximity sensors may be employed to provide more DOF's. As an alternative to a single proximity sensor, a force sensor integrated in a headgear strap (e.g., strap 22) securing mask 14 to the face of patient P can instead be used to provide a simple model input (an imposed force), thus eliminating the need for location and orientation sensing. In such example, sensor data may be streamed directly to computing arrangement 28 or via a CPAP machine used in providing PAP treatment session 12.

In another example embodiment of the present invention, computational model 30 includes a 3D finite element (FE) model that simulates mask 14 in contact with the head of patient P based on the real-time relative position data obtained by sensing arrangement 24 and calculates the skin pressure, slip (or shear) and gap distribution between the mask seal or cushion 18 and the face of patient P. In such example the geometry of mask 14 is obtained from a CAD file or other suitable source. The patient specific face geometry is obtained from a 3D facial scan (e.g., acquired during mask fit, PSG nights, etc.) or other suitable source. As a boundary condition, the model takes as real time input either a measured displacement or a force such as previously described. For most mask designs the shell can be assumed rigid. Gravity can be included as well; dependent on the particular mask (e.g., masks of greater than negligible weight) this is potentially important when the patient's head is in a lateral position and the weight of the mask exerts shear on slack skin. Because a 3D simulation requires significant time, a real time full simulation might be not feasible. To solve this, a reduced order model that requires low computational power, data storage and energy consumption, may be utilized (at the expense of less detailed results). Depending on the available computational resources and model complexity, the finite element model can be evaluated in real time or intermittently, for example, when patient P changes position, or once a day when mask 14 has been set-up for the night on patient P.

In the aforementioned example embodiments, the output from computational model 30 is a file containing time-based information regarding one or more of skin pressure distribution, slip (or shear) distribution, and/or gap distribution. Such output is communicated to interpretation and analysis unit 32. The model output itself, i.e. the skin pressure, shear and gap distribution, is not directly informative for a patient or a physician. The model output needs to be interpreted in terms of skin comfort, leakage and mask stability. For this purpose, the model output can be compared with threshold values, for example, without limitation: a maximum skin pressure to determine the risk for skin breakdown, e.g. derived from the patient's blood pressure (e.g., provided by the patient or physician, actively obtained by a sensor, etc.); a zero gap along the circumference of the contact between the seal or cushion and the face, to determine if there is an unintentional leakage for example near the eyes (see also the example embodiment below regarding leak flow sensing and modeling); and a zero slip between the seal or cushion and the face to determine if the mask remains stable. Comparing the longitudinal (i.e., time-based) model output with these threshold values provides regional, temporal and contextual information on potential problems, i.e., the nature of a potential problem (skin pressure, leak, slip), when it occurs (time, body position, situational context) and where it occurs (mouth, nose, eyes, cheek, forehead). In addition to such quantitative comparison between model predictions and threshold values quantitative trend information might also be relevant. Based on comparing the output from computational model 30 with threshold values, risk estimations and root causes are determined, such as, for example, without limitation: a probability for skin breakdown due to strap force, mask fit, body position, seal stiffness, moisture (sweat, humidity), wear-time etc.; a probability for leakage due to CPAP pressure, tossing and turning, strap force, a dirty mask, particles/foreign pieces between mask and skin, an old mask, sleep position, etc.; a probability for slip due to strap forces, tossing and turning, mask fit, seal friction, moisture, etc. The simplest probability scale is risk/no risk.

The value of the model based information can be increased even further when combined with additional data such as CPAP device data (pressure, leak flow, AHI events, arousals), therapy outcomes (patient fitness), patient body position, mask lifetime, mask cleaning data, temperature and humidity etc. Machine learning algorithms (regression, deep learning, patient similarity networks etc.) implemented in computing arrangement 28 can detect relations between these additional data, the model input and output, patient subtypes and the therapy outcomes. These machine learning detected relations provide an even richer insight into how a mask influences the therapy compliance and effectiveness for specific patients or groups of patients.

In advanced embodiments of the present invention actionable information and insights communicated to the patient and/or other persons may be in the form of warnings or recommendations such as:
There is an increased risk for skin breakdown: loosen your headgear or choose a less snug fit, clean mask, switch mask.
The reason for your recent nocturnal arousals or your increased residual AHI events is the leakage near your eyes during the sleep phase while sleeping at your side.
Consider a replacement mask that avoids leakage near your eyes.
The mask lifetime has expired so buy a new mask because this one starts to leak and causes an increased residual AHI.
Clean your mask because your low cleaning adherence causes leakage and slip on your cheek due to which the CPAP pressure is not sufficiently anymore to keep your airways open and/or creates skin damage.
Positional therapy is recommended because your frequent lateral body position causes leakage towards the eyes which causes arousals.

In other example embodiments in accordance with the present invention, the output from computational model 30 is used to control one or more strap actuators that are each structured to adjust tension in one or more straps (e.g., straps 20 and 22) used to secure mask 14 to the head of a patient in order to optimize the mask function (e.g., no red marks, no unintended leakage, stable mask).

In other example embodiments, data gathered by a sleep therapy device (e.g., without limitation, a CPAP machine) is used to determine if/when sensing of mask 14 and the related actions carried out by computing arrangement 28 are carried out. For example, sleep therapy devices typically report and/or display total leak rates. The total leak is the intentional leak of the exhalation ports (generally in the order of 100-1000 ml/s depending on the mask design and CPAP pressure) plus the unintentional leak (from mouth, mask, etc.). The unintentional mask leakage becomes a problem when air is rushing into the eyes, when the leakage is excessive such that the therapy pressure cannot be maintained, or when the sound of the escaping air otherwise disturbs the patient or a bed partner of the patient. From the measured total leak rate, the unintentional leak rate can be estimated, but it cannot distinguish the source, location and impact of the unintentional leak. In some cases, the unintentional leak rate might be small so that it remains undetected. For example, a relatively small leakage of a few ml/s into the eyes may already cause the patient to be disturbed or aroused.

By using a computational fluid dynamics (CFD) simulation—or a simpler analytical model—the leak flow through a non-zero gap can be calculated. Boundary conditions are for example the CPAP pressure inside—and ambient pressure outside the mask. The calculated leak flow rate can be compared with a location dependent threshold, for example 1 ml/s at the nose bridge. The measured unintentional leak rate can be used to decide on when there is a need for virtual sensing of the relative positioning of mask 14 with the face of patient P. For example, only when the unintended leak is above a certain threshold, e.g. above the detection limit or above e.g. >10 ml/sec, the virtual sensor is activated to search for the leak location. In another related example, the virtual sensor is activated if a patient provides an indication (e.g., via application 34 of user device 36) of a notable (and likely undesirable) leak (e.g., near the eyes, disturbing a bed partner, etc.).

In another example embodiment of the present invention, sensing arrangement 24 includes a number of thermal sensors for leak detection. In a majority of instances where a PAP treatment is provided, exhaled breath from patient P and or heated/humidified air provided in the PAP treatment is/are warmer than the environmental temperature. By placing thermistors on specific spots around (and on the outside of) mask 14 (e.g., away from the contact surface and in a position so as to not adversely affect the sealing portion), small temperature changes around the edge of mask 14 can be measured/monitored. Such arrangement not only provides for leak detection in general (even in real-time), but also can provide a specific location for the leak as well as the leak rate. Such information can be used to improve the accuracy and feedback provided by system 10. Additionally, information from such sensors can provide an accurate representation of the quality of the seal, displacements that compromise it, or even degradation of the seal/interface due to deterioration of the mask seal (which in turn could trigger the advice to the user to replace the mask as discussed above). Instead of (or in addition to) sensors placed around the mask, an infrared (IR) camera or cameras may be provided in sensing arrangement 24. In related embodiments a stereo microphone or multiple microphones are used to localize the sound created by the air turbulences of leaking air. The loudness of a leak can be related to the leak rate based on initial reference measurements.

From the foregoing examples, it is thus to be appreciated that embodiments of the present invention overcome shortcomings of the prior art by providing arrangements that improve the interaction between the mask and the patient when the patient is receiving further PAP treatment via the mask. It is also to be appreciated that embodiments of the present invention may be applied generally to any to other applications wherein pressurized air and/or supplementary oxygen is provided to a patient via a mask.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A system (10) for monitoring the interaction of a mask (14) with the face of a patient in real-time during a pressure therapy treatment session (12) and providing recommendations for improving such interaction, the system comprising:
a sensing arrangement (24) structured to obtain real-time relative position data regarding the relative position of the mask relative to face of the patient; and
a computing arrangement (28) in communication with the sensing arrangement for receiving the real-time relative position data from the sensing arrangement, the computing arrangement comprising:
a computational model (30) structured to use the real-time relative position data to determine information regarding the interaction between the mask and the face of the patient, and
an interpretation and analysis unit (32) structured to relate the information regarding the interaction between the mask and the face of the patient to functional parameters, to determine actionable information and/or insights to improve the interaction, and to provide as output the actionable information and/or insights.

2. The system of claim 1 further comprising a user device (36) executing an application (34) for receiving the output from the interpretation and analysis unit.

3. The system of claim 2, wherein the user device comprises a smartphone.

4. The system of claim 1, wherein the information regarding the interaction between the mask and the face of the patient comprises one or more of: a skin pressure distribution resulting from the mask being pressed against the face of the patient, a gap distribution of a number of gaps between the mask and the face of the patient, and/or a slip distribution of the mask relative to the face of the patient.

5. The system of claim 1, wherein the functional parameters comprise one or more of: mask comfort, mask seal, and/or mask stability.

6. The system of claim 1, wherein the sensing arrangement comprises a camera system.

7. The system of claim 1, wherein the sensing arrangement comprises a number of physical sensors.

8. The system of claim 1, wherein the sensing arrangement comprises a number of thermal sensors.

9. The system of claim 1, wherein the sensing arrangement comprises a number of microphones.

10. The system of claim 1, wherein the computational model utilizes a virtual model of the mask that is positioned, based on the real-time relative positon data, with respect to a virtual head and/or face model that is specific to the patient.

11. The system of claim 1, wherein the interpretation and analysis unit comprises a neural network.

12. The system of claim 1, wherein the computing arrangement is a cloud-based computing arrangement.

13. A method (40) for improving the interaction between of a mask used in a pressure therapy treatment session and a face of a patient receiving the pressure therapy, the method comprising:
obtaining (42) real-time relative position data of the mask relative to the face of the patient while the patient is receiving the pressure therapy via the mask;
determining (44) information regarding the interaction between the mask and the face of the patient;
determining (46) one or more of: comfort, seal, and/or stability of the mask that can be improved is determined from the information regarding the interaction; and
determining (48) actionable information to improve the one or more of comfort, seal, and or stability of the mask on the face of the patient.

14. The method of claim 13, further comprising one or both of:
providing (50) the actionable information to the patient or a caregiver of the patient; and/or
carrying out the actionable information.

15. The method of claim 13, wherein determining (44) information regarding the interaction between the mask and the face of the patient comprises determining one or more of: a skin pressure distribution, a gap distribution, and/or a slip distribution.
